(19) United States
(12) Patent Application Publication
(10) Pub. No.: US 2024/0285176 A1
(43) Pub. Date: Aug. 29, 2024

(54) HEAT DETECTOR, HEAT DETECTION METHOD AND PROGRAM

(71) Applicant: Nippon Telegraph and Telephone Corporation, Tokyo (JP)

(72) Inventors: Yuki Hashimoto, Tokyo (JP); Kazuhiko Takagahara, Tokyo (JP); Hiroyoshi Togo, Tokyo (JP)

(21) Appl. No.: 18/689,739

(22) PCT Filed: Oct. 22, 2021
(86) PCT No.: PCT/JP2021/039058

§ 371 Date: Mar. 6, 2024

(51) Int. Cl. A61B 5/0205; A61B 5/024; A61B 5/083; A61B 5/11; G01P 15/18

(52) U.S. Cl. CPC A61B 5/02055; A61B 5/0833; A61B 5/1118; A61B 5/1126; G01P 15/18; A61B 5/024; A61B 2562/0219

(57) ABSTRACT

A fever detection device includes a heart rate measurement unit configured to measure a heart rate of a target subject; an acceleration measurement unit configured to measure an acceleration of the target subject; an exercise intensity calculation unit configured to calculate exercise intensity of the target subject based on the acceleration; and a fever detection unit configured to determine whether the target subject has a fever based on the heart rate and the exercise intensity.

### CROSS-REFERENCE TO RELATED APPLICATIONS

This application is a national phase entry of PCT Application No. PCT/JP2021/039058, filed on Oct. 22, 2021, which application is hereby incorporated herein by reference.

### TECHNICAL FIELD

The present invention relates to a fever detection device, a fever detection method and a program, each of which is capable of detecting that a person has a fever.

### BACKGROUND

In recent years, the importance of screening for people with a fever has increased due to the spread of the new coronavirus infection. For example, facilities with thermography or spot thermometers have increased, and skin temperature distributions of the entire body, a specific region or a specific spot of a facility user are measured and used for screening for a person with a fever (see Non Patent Literature 1).

In general, when the core body temperature of a person rises, skin blood flow increases in order to increase the efficiency of heat dissipation from the skin. Therefore, the higher the core body temperature is, the higher the skin temperature tends to be. However, the skin temperature of a person changes from time to time due to various external factors such as core body temperature, ambient environmental temperature, clothes worn by a target subject, solar radiation to the skin, and sweat evaporation. Accordingly, the monitoring of the skin temperature by a thermography or spot thermometer has a problem that information reflecting only the body temperature rise is not always obtained.

### CITATION LIST

### Non Patent Literature

Non Patent Literature 1: Masafumi Kimata, “Non-contact Body Temperature Measurement”, Special WEB Column, “Applied Physics Learned from New Coronavirus Pandemic”, The Japan Society of Applied Physics, 2020, <https://www.jsap.or.jp/docs/columns-covid19/covid19_3-1.pdf>

### SUMMARY

### Technical Problem

Embodiments of the present invention have been made to solve the problems above, and an object thereof is to provide a fever detection device, a fever detection method and a program, each of which is capable of determining whether a target subject has a fever without being affected by external factors such as ambient environmental temperature.

### Solution to Problem

A fever detection device according to embodiments of the present invention includes: a heart rate measurement unit configured to measure a heart rate of a target subject; an acceleration measurement unit configured to measure an acceleration of the target subject; an exercise intensity calculation unit configured to calculate exercise intensity of the target subject based on the acceleration; and a fever detection unit configured to determine whether the target subject has a fever based on the heart rate and the exercise intensity.

In one configuration example of the fever detection device according to embodiments of the present invention, the acceleration measurement unit is configured to measure a uniaxial acceleration or a triaxial acceleration of the target subject.

In one configuration example of the fever detection device according to embodiments of the present invention, the fever detection unit is configured to determine that the target subject has a fever in a case where a difference between the heart rate and a heart rate corresponding to the exercise intensity exceeds a threshold.

In one configuration example of the fever detection device according to embodiments of the present invention, the fever detection unit is configured to calculate a heart rate corresponding to the exercise intensity based on an at-rest heart rate, a maximum heart rate, an at-rest oxygen uptake, and a maximum oxygen uptake, all of which are known for the target subject, as well as the exercise intensity.

In one configuration example of the fever detection device according to embodiments of the present invention, the fever detection unit is configured to calculate the threshold based on a tolerable body temperature, an at-rest normal body temperature, and a heart rate increase value with respect to an increase in a body temperature, of the target subject.

In one configuration example of the fever detection device according to embodiments of the present invention, the fever detection unit is configured to set the tolerable body temperature in accordance with whether the target subject has heat acclimation.

A fever detection method according to embodiments of the present invention includes: a first step of measuring a heart rate of a target subject; a second step of measuring an acceleration of the target subject; a third step of calculating exercise intensity of the target subject based on the acceleration; and a fourth step of determining whether the target subject has a fever based on the heart rate and the exercise intensity.

A fever detection program according to embodiments of the present invention is a program causing a computer to execute each step described above.

### Advantageous Effects

According to embodiments of the present invention, by providing the heart rate measurement unit, the acceleration measurement unit, the exercise intensity calculation unit, and the fever detection unit, it is possible to determine whether the target subject has a fever without being affected by external factors such as ambient environmental temperature, clothes worn by the target subject, solar radiation to the skin, and sweat evaporation.

### BRIEF DESCRIPTION OF THE DRAWINGS

FIG. 1 is a block diagram illustrating a configuration of a fever detection device according to an example of the present invention.

FIG. 2 is a flowchart illustrating an operation of the fever detection device according to an example of the present invention.

FIG. 3 is a block diagram illustrating a configuration example of a computer that implements the fever detection device according to an example of the present invention.

### DETAILED DESCRIPTION OF ILLUSTRATIVE EMBODIMENTS

In embodiments of the present invention, a heart rate and an acceleration of a target subject are measured, and exercise intensities estimated from two measurements are compared to detect whether the target subject has a fever.

### Example

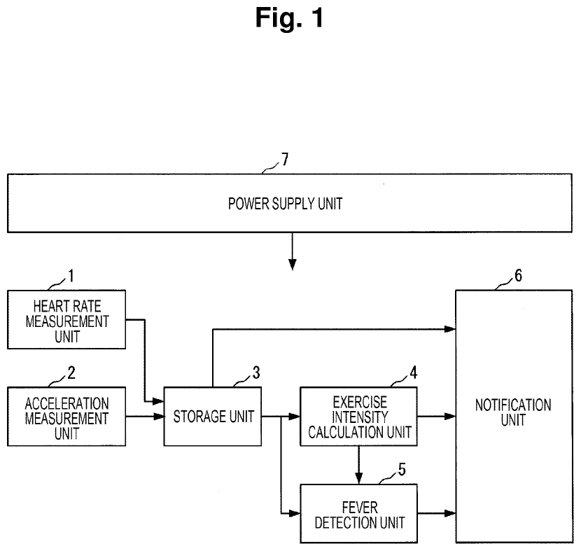

Hereinafter, examples of the present invention will be described with reference to the drawings. FIG. 1 is a block diagram illustrating a configuration of a fever detection device according to an example of the present invention. The fever detection device includes a heart rate measurement unit 1, an acceleration measurement unit 2, a storage unit 3, an exercise intensity calculation unit 4, a fever detection unit 5, a notification unit 6, and a power supply unit 7.

The heart rate measurement unit 1 measures a heart rate HR of the target subject. The heart rate measurement unit 1 includes, for example, a wearable or belt-type electrocardiograph that measures an electrocardiogram of the target subject, and a calculation unit that calculates the heart rate from the electrocardiogram measured by the electrocardiograph. In embodiments of the present invention, a pulse rate of the target subject may be measured as the heart rate. In this case, the heart rate measurement unit 1 includes a wristband-type or earphone-type pulse wave meter that measures a pulse wave of the target subject, and a calculation unit that calculates a heart rate (pulse rate) from the pulse wave measured by the pulse wave meter.

The acceleration measurement unit 2 is attached to the body of the target subject and measures acceleration A of one axis or a plurality of axes of the target subject.

The heart rate measurement unit 1 and the acceleration measurement unit 2 may be an integrated device or may be individual devices.

The storage unit 3 stores time-series data of the heart rate HR measured by the heart rate measurement unit 1 and time-series data of the acceleration A measured by the acceleration measurement unit 2. The storage unit 3 is implemented by, for example, a non-volatile memory represented by a flash memory, or a volatile memory such as a dynamic random access memory (DRAM).

The exercise intensity calculation unit 4 calculates the exercise intensity of the target subject from the acceleration A measured by the acceleration measurement unit 2, and outputs the time-series data of the exercise intensity. A specific example of an index representing exercise intensity is metabolic equivalents (METs).

The exercise intensity calculation unit 4 performs filter processing on the triaxial accelerations Ax, Ay and Az measured by the acceleration measurement unit 2 to remove a gravitational acceleration component, and then calculates a combined acceleration of the three axes, and estimates METs from the combined acceleration by a regression equation. This method is disclosed in K. Ohkawara, et. al., Real-time estimation of daily physical activity intensity by a triaxial accelerometer and a gravity-removal classification algorithm, British Journal of Nutrition, Vol. 105, pp. 1681-1691, 2011.

Additionally, the exercise intensity calculation unit 4 estimates the METs from a known relationship between a count value calculated from the uniaxial acceleration A measured by the acceleration measurement unit 2 and the METs. This method is disclosed in S. H. Kozey, et. al., Accelerometer Output and MET Values of Common Physical Activities, Medicine & Science in Sports & Exercise, Vol. 42, pp. 1776-1784, 2010.

The method is not limited to the method above, and the METs may be estimated from the acceleration A using another method.

The fever detection unit 5 determines whether the target subject has a fever based on the heart rate HR measured by the heart rate measurement unit 1 and the exercise intensity (METs) calculated by the exercise intensity calculation unit 4. In particular, the fever detection unit 5 determines that the target subject has a fever in a case where a difference HR−HRₑᵥ between the heart rate HR and a heart rate HRₑᵥ corresponding to METs exceeds a preset threshold TH. The heart rate HRₑᵥ corresponding to METs can be calculated by, for example, the following equation.

$$\begin{matrix}Equation 1 & \\ {HR}_{ev} = {HR}_{rest} + METs \times \frac{{VO}_{2 rest}}{{VO}_{2 max}} \times ( {HR}_{max} - {HR}_{rest} ) & ( 1 )\end{matrix}$$

In Equation (1), HRᵣₑₛₜ [bpm] is an at-rest heart rate of the target subject, HRₘₐₓ [bpm] is a maximum heart rate of the target subject, VO₂ᵣₑₛₜ [mL] is an at-rest oxygen uptake of the target subject, and VO₂ₘₐₓ [mL] is a maximum oxygen uptake of the target subject. As the at-rest heart rate HRᵣₑₛₜ, the maximum heart rate HRₘₐₓ, the at-rest oxygen uptake VO₂ᵣₑₛₜ, and the maximum oxygen uptake VO₂ₘₐₓ, actual values may be set in advance as known values obtained by previous measurement.

Further, the fever detection unit 5 may estimate the heart rate HRₑᵥ from the METs calculated by the exercise intensity calculation unit 4 using a relationship between the heart rate and the METs disclosed in J. R. Wicks, et al., HR Index—A Simple Method for the Prediction of Oxygen Uptake, Medicine and Science in Sports and Exercise, 2011.

The fever detection unit 5 may calculate the threshold TH [bpm] by, for example, the following equation.

$$TH = ( {T}_{LV} - {T}_{rest} ) \times {HR}_{I} \qquad ( 2 )$$

T_{LV} [° C.] is a tolerable body temperature of the target subject, Tᵣₑₛₜ [° C.] is an at-rest normal body temperature of the target subject, and HR₁ [bpm/° C.] is a heart rate increase value with respect to an increase in the body temperature of the target subject. As the tolerable body temperature T_{LV} [° C.], a tolerable body temperature during activity under heat disclosed in Brenda Jacklitsch, et al., Occupational exposure to heat and hot environments, US Department of Health and Human Services, Centers for Disease Control and Prevention, NIOSH:Cincinnati, OH, USA, 2016:1-159, <https://www.cdc.gov/niosh/docs/2016-106/pdfs/2016-106.pdf?id=10.26616/NIOSHPUB2016106>” can be used. The tolerable body temperature disclosed in this reference varies depending on whether the target subject is subjected to heat acclimation. Therefore, the fever detection unit 5 may set the tolerable body temperature T_{LV} [° C.] according to whether the target subject has heat acclimation. Information on whether the target subject has heat acclimation is input in advance to the fever detection device by the target subject or a third party.

In addition, a reference value for fever or the reference value for rising fever disclosed in the Ministry of Health, Labour and Welfare, Standards for Which Practitioners and Managers of Designated Reporting Organizations Make a Reports to Prefectural Governors <https://www.mhlw.go.jp/content/10900000/000788099.pdf> may be a known tolerable body temperature T_{LV} [° C.].

As the heart rate increase value HR₁ [bpm/° C.] with respect to the increase in the body temperature, a known value disclosed in H. K. Walker, W. D. Hall, J. W. Hurst, Clinical Methods: The History, Physical, and Laboratory Examinations, 3rd edition, Boston: Butterworths, 1990, Chapter 218, <https://www.ncbi.nlm.nih.gov/books/NBK331/> may be used, or a known value obtained by previous measurement may be used. For the at-rest normal body temperature Tᵣₑₛₜ [° C.], an actual value may be set in advance as a known value obtained by previous measurement.

The notification unit 6 transmits the heart rate HR measured by the heart rate measurement unit 1, the acceleration A measured by the acceleration measurement unit 2, the METs calculated by the exercise intensity calculation unit 4, and the determination result of the fever detection unit 5 to an external device (not illustrated) such as a smartphone in a wireless or wired manner.

Examples of the wireless communication protocol include Bluetooth (registered trademark) Low Energy (BLE). Examples of the wired communication protocol include Ethernet (registered trademark).

The power supply unit 7 serves as a circuit that supplies power to the fever detection device.

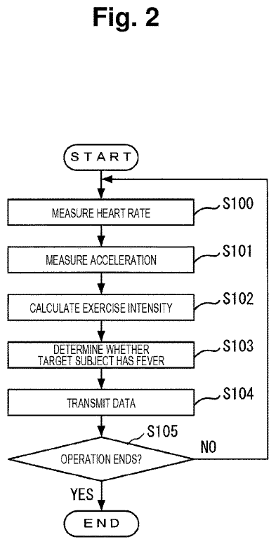

FIG. 2 is a flowchart for describing an operation of the fever detection device of the present example. The heart rate measurement unit 1 measures a heart rate HR[t] of the target subject at a time t (step S100 in FIG. 2). The acceleration measurement unit 2 measures an acceleration A[t] of the target subject at the time t (step S101 in FIG. 2).

The exercise intensity calculation unit 4 calculates METs[t] of the target subject at the time t from the acceleration A [t] (step S102 in FIG. 2).

The fever detection unit 5 determines whether the target subject has a fever based on the heart rate HR[t] and the METs[t] (step S103 in FIG. 2). Specifically, the fever detection unit 5 calculates the heart rate HRₑᵥ[t] of the target subject corresponding to the METs at the time t by Equation (1). Then, the fever detection unit 5 determines that the target subject has a fever in a case where a difference HR[t]−HRₑᵥ[t] between the heart rate HR[t] and HRₑᵥ[t] exceeds the threshold TH, and determines that the target subject does not have a fever in a case where the difference HR[t]−HRₑᵥ[t] is equal to or less than the threshold TH.

The notification unit 6 transmits the heart rate HR[t], the acceleration A[t], the METs[t], and the determination result of the fever detection unit 5 to an external device in a wireless or wired manner (step S104 in FIG. 2).

The fever detection device repeatedly executes the processing of steps S100 to S104, for example, until there is an instruction to end the operation from the target subject (YES in step S105 in FIG. 2).

As described above, according to the present example, it is possible to determine whether the target subject has a fever without being affected by external factors such as ambient environmental temperature, clothes worn by the target subject, solar radiation to the skin, and sweat evaporation.

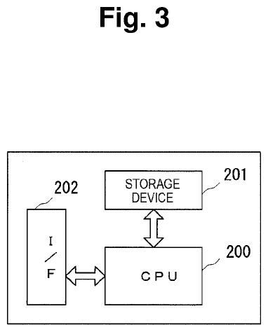

The storage unit 3, the exercise intensity calculation unit 4, the fever detection unit 5 and the notification unit 6 described in the present example can be implemented by a computer including a central processing unit (CPU), a storage device, and an interface and a program for controlling those hardware resources. A configuration example of this computer is illustrated in FIG. 3.

The computer includes a CPU 200, a storage device 201, and an interface device (I/F) 202. Hardware of the heart rate measurement unit 1, the acceleration measurement unit 2, and the notification unit 6 is connected to the I/F 202. In such a computer, a fever detection program for implementing the fever detection method of embodiments of the present invention is provided in a state of being recorded on a recording medium such as a flexible disk, a CD-ROM, a DVD-ROM, or a memory card. The CPU 200 writes a program read from the recording medium into the storage device 201, and executes the processing described in the present example according to the program stored in the storage device 201. The fever detection program may also be provided via a network.

### INDUSTRIAL APPLICABILITY

Embodiments of the present invention can be applied to a method for detecting whether a person has a fever in a non-contact manner.

### REFERENCE SIGNS LIST

1 Heart rate measurement unit
2 Acceleration measurement unit
3 Storage unit
4 Exercise intensity calculation unit
5 Fever detection unit
6 Notification unit
7 Power supply unit

1.-8. (canceled)

9. A device, comprising:
a heart rate measurement device configured to measure a heart rate of a target subject;
an acceleration measurement device configured to measure an acceleration of the target subject;
a memory storage storing instructions; and
one or more processors in communication with the memory storage, wherein the one or more processors execute the instructions to:
calculate exercise intensity of the target subject based on the acceleration of the target subject; and
determine whether the target subject has a fever based on the heart rate of the target subject and the exercise intensity of the target subject.

10. The device according to claim 9, wherein the acceleration measurement device is configured to measure a uniaxial acceleration or a triaxial acceleration of the target subject.

11. The device according to claim 9, wherein the instructions to determine whether the target subject has a fever include instructions to determine that the target subject has a fever in response to a difference between the heart rate of the target subject and a heart rate corresponding to the exercise intensity exceeding a threshold.

12. The device according to claim 11, wherein the instructions include further instructions to calculate the heart rate corresponding to the exercise intensity based on an at-rest heart rate of the target subject, a maximum heart rate of the target subject, an at-rest oxygen uptake of the target subject, and a maximum oxygen uptake of the target subject as well as the exercise intensity.

13. The device according to claim 11, wherein instructions include further instructions to calculate the threshold based on a tolerable body temperature of the target subject, an at-rest normal body temperature of the target subject, and a heart rate increase value with respect to an increase in a body temperature of the target subject.

14. The device according to claim 13, wherein the instructions include further instructions to set the tolerable body temperature in accordance with whether the target subject has heat acclimation.

15. A method, comprising:
a first step of measuring a heart rate of a target subject;
a second step of measuring an acceleration of the target subject;
a third step of calculating exercise intensity of the target subject based on the acceleration of the target subject; and
a fourth step of determining whether the target subject has a fever based on the heart rate of the target subject and the exercise intensity of the target subject.

16. The method according to claim 15, wherein the second step comprises measuring a uniaxial acceleration or a triaxial acceleration of the target subject.

17. The method according to claim 15, wherein the fourth step comprises determining that the target subject has a fever in response to a difference between the heart rate of the target subject and a heart rate corresponding to the exercise intensity exceeding a threshold.

18. The method according to claim 17, further comprising a fifth step of calculating the heart rate corresponding to the exercise intensity based on an at-rest heart rate of the target subject, a maximum heart rate of the target subject, an at-rest oxygen uptake of the target subject, and a maximum oxygen uptake of the target subject as well as the exercise intensity.

19. The method according to claim 17, further comprising a sixth step of calculating the threshold based on a tolerable body temperature of the target subject, an at-rest normal body temperature of the target subject, and a heart rate increase value with respect to an increase in a body temperature of the target subject.

20. The method according to claim 19, further comprising a seventh step of setting the tolerable body temperature in accordance with whether the target subject has heat acclimation.

21. A non-transitory computer-readable storage device storing a fever detection program that when executed by one or more processors, cause the one or more processors to execute:
a first step of measuring a heart rate of a target subject;
a second step of measuring an acceleration of the target subject;
a third step of calculating exercise intensity of the target subject based on the acceleration of the target subject; and
a fourth step of determining whether the target subject has a fever based on the heart rate of the target subject and the exercise intensity of the target subject.

22. The non-transitory computer-readable storage device according to claim 21, wherein the second step comprises measuring a uniaxial acceleration or a triaxial acceleration of the target subject.

23. The non-transitory computer-readable storage device according to claim 21, wherein the fourth step comprises determining that the target subject has a fever in response to a difference between the heart rate of the target subject and a heart rate corresponding to the exercise intensity exceeding a threshold.

24. The non-transitory computer-readable storage device according to claim 23, wherein the fever detection program that when executed by one or more processors, further cause the one or more processors to execute:
a fifth step of calculating the heart rate corresponding to the exercise intensity based on an at-rest heart rate of the target subject, a maximum heart rate of the target subject, an at-rest oxygen uptake of the target subject, and a maximum oxygen uptake of the target subject as well as the exercise intensity.

25. The non-transitory computer-readable storage device according to claim 23, wherein the fever detection program that when executed by one or more processors, further cause the one or more processors to execute:
a sixth step of calculating the threshold based on a tolerable body temperature of the target subject, an at-rest normal body temperature of the target subject, and a heart rate increase value with respect to an increase in a body temperature of the target subject.

26. The non-transitory computer-readable storage device according to claim 25, wherein the fever detection program that when executed by one or more processors, further cause the one or more processors to execute:
further comprising a seventh step of setting the tolerable body temperature in accordance with whether the target subject has heat acclimation.
